(19) United States
(12) Patent Application Publication
(10) Pub. No.: US 2025/0391574 A1
(43) Pub. Date: Dec. 25, 2025

(54) METHOD OF DETECTING A CHANGE IN ELECTRICAL SIGNALS IN A BRAIN OF A USER USING EEG

(71) Applicant: UNIVERSITEIT LEIDEN, Leiden (NL)

(72) Inventor: Arko GHOSH, Leiden (NL)

(73) Assignee: UNIVERSITEIT LEIDEN, Leiden (NL)

(21) Appl. No.: 19/268,407

(22) Filed: Jul. 14, 2025

Related U.S. Application Data
(62) Division of application No. 17/612,866, filed on Nov. 19, 2021, now abandoned.

(30) Foreign Application Priority Data
May 22, 2019 (NL) 2023177
May 24, 2019 (NL) 2023196

(51) Int. Cl. G16H 50/30; A61B 5/00

(52) U.S. Cl. CPC G16H 50/30; A61B 5/4064; A61B 5/6898

(57) ABSTRACT

Described herein is a method of detecting a change in electrical signals in a brain of a user, the method comprising at least recording at least one electrical signal in the brain of a user by EEG; and recording at least one user behavior comprising a tap, a swipe, a gesture, or a sound input; wherein the recording is performed while the user is using a portable electronic device.

### CROSS-REFERENCE TO RELATED APPLICATIONS

This application is a divisional under 35 U.S.C. § 121 of co-pending U.S. application Ser. No. 17/612,866 filed Nov. 19, 2021, which is a 35 U.S.C. § 371 National Phase Entry Application of International Application No. PCT/NL2020/050328 filed May 22, 2020 the contents of which are incorporated by reference herein in their entirety, and which claims benefit under 35 U.S.C. § 119(a) of NL Application Nos. 2023177 filed May 22, 2019 and 2023196 filed May 24, 2019.

### TECHNICAL FIELD

The present invention relates to a method and a system for analysing functioning of a brain of a user, such as a user of a portable electronic device. The present invention also relates to the use of a portable electronic device, such as with a touchscreen, to analyse the functioning of a brain of a user of the device.

### BACKGROUND

Smartphones require just a few gestures by the user on the screen to operate it—mainly taps and swipes, and this allows the user to participate in a broad range of activities. According to recent estimates, young adults generate about 4000 touchscreen touches per day. For voluntary, self-paced motor control (“VSPMC”) of a button press by a finger of an individual, neuronal processing by the individual's brain can begin 1.5-2 seconds before the onset of movement of the finger (Shibasaki, H. & Hallett, M., (2006) What is the Bereitschaftspotential? Clin. Neurophysiol. 117, 2341-2356. The resultant brains' electrical signal, when measured from the scalp (e.g. Electroencephalogram (EEG)), is negative and emerges gradually over time to peak when the button is depressed. This widely studied signal has been separated into: the readiness potential (“RP”), the motor potential (“MP”) and the reafferent potential (“RAP”) which reflect the different stages of motor control as in movement preparation, execution and processing of the resultant proprioceptive information respectively Shibasaki, H. & Hallett, M., (2006) What is the Bereitschaftspotential? Clin. Neurophysiol. 117, 2341-2356. The RP is considered central to higher brain functions and may hold the biological marker for wilful action initiation. The later MP and RAP are considered to be closely involved in generating motor cortical outputs and monitoring the ongoing movement. However, these empirical insights cannot be simply applied to smartphones. For instance, a pair of touches is typically separated by less than 500 milliseconds on a touchscreen of a smartphone which is far shorter than the normal preparatory time observed for the self-paced button presses. Moreover, the touches have a range of consequences—from shopping to dating—in contrast to the near constant outcomes of laboratory button presses. Thus, the time-course of neuronal signals underlying an individual's smartphone movements remains unclear.

Correlations between neuronal activity and behavioural outputs are identified by synchronous neuronal recording and behavioural output recording, such as with at least two devices with synchronised clocks whereby outputs from each device are plotted along a single time axis.

### SUMMARY

The continuous monitoring of neuronal activity at a sub-second resolution over broad spans of time offers fresh opportunities to understand and evaluate brain functions. The emergence of mobile EEG enables long periods of neuronal recordings out of the laboratory. Not just that implanted electrodes in the brain can gather neuronal data for years. One substantial obstacle in the analysis of these brain signals is that there is no high-resolution temporal land mark in day-to-day behaviour. In contrast, conventional laboratory-based measures can powerfully leverage land markers such as in visual evoked potentials->the timing of the artificial visual stimuli, somatosensory evoked potentials->the timing of artificial shocks or touches, and motor related potentials->the timing of instructed button presses.

One unexplored avenue for enabling event-based analysis of brain signals when engaged in spontaneous behaviour is to exploit the high-resolution smartphone touchscreen events.

However, in spite of this access the avenue is not without barriers: (a) It is not known whether the touchscreen interactions have any consistent neuronal activity patterns associated with them and (b) Typical event-related analysis depends on synchronizing independent clocks to a millisecond resolution prohibiting a seamless analysis of the neurobehavioral data even if the smartphone events were collected and if the consistent patterns were known.

The present inventor's research has identified that previous work based on laboratory-based tasks offers some reasonable expectations on the nature of the neuronal signals surrounding smartphone interactions. For example, the present inventor has identified that US2017/351958 (in the names of Universitat Zurich and University of Fribourg) relies upon a regression model and merely provides a “predicted brain response” to a user. US2017/351958 is concerned with estimating a “brain state” and allowing a user to decide to alter device use (by self-regulation) if desired. US2017/351958 explicitly requires a “sensory stimulus”; and teaches that a plurality of usage data sets from a plurality of different users is an essential feature for generating a computational interference model. However, the present inventor here has novelly developed for this present application, methods that enable the determination of a user's event-related neuronal activation upon a comparison with predefined behavioural outputs associated with known event-related neuronal activations. The present inventor has uniquely identified that such determination can be performed in loco, outside a laboratory, without requiring any stimulus, as described in detail herein. The present inventor has even originally identified the ability to utilise the monitoring of in loco behavioural outputs to determine change in a user's neuronal activity over a period of time, sequentially, to identify remotely a development in the user's neuronal activity.

Theoretical and empirical work across various neurosciences suggests that neuronal circuits of the brain, typically considered as engaged in the details of VSPMC in an individual, are crucial factors in higher cognition in the individual, as well as in the individual's social interactions and emotions. For instance, the MP of an individual is depressed by an increased cognitive load with no overt motor impact and emotionally laden stimuli depress MP related signals. The effects can be explained by the simultaneous engagement of diverse neuronal processes intertwined with sensorimotor processing in cognitively demanding situations.

Theoretical and empirical work also suggests that event-based analysis of brain signals with a focus on sensory events can reveal various aspects of brain functions in health and diseases. For instance, the laboratory-derived visual evoked potentials are routinely deployed to access visual processing abnormalities in multiple sclerosis, stroke or epilepsy.

However, there has been an unfilled need for a method and system for better analysing the functioning of such neuronal circuits of the brain; such as where they are engaged in an individual's voluntary, self-paced motor control with a range of possible behavioural outcomes (e.g. of a button press with a finger, or the like). There is also an unfilled need for a method and system that capture multiple parts of the neuronal processing simultaneously without needing dedicated tasks or tests.

According to an aspect of the present disclosure there is provided a method of monitoring a user's neuronal activity. The method may comprise recording the user's behavioural output outside of a laboratory; and/or spontaneous behaviour with limited instructions in the laboratory (such as in ‘use your smartphone to check your messages’). The method may comprise recording the user's behavioural output in loco and/or in a laboratory. The method may comprise recording the user's behavioural output unobtrusively. The method may comprise recording the user's behavioural output in loco. The method may comprise recording the user's behavioural output without simulation, such as without artificial simulation. For example, the method may comprise recording the user's behavioural output during an unprompted, natural activity/ies. The method may comprise recording the user's behavioural output without stimulation, such as without artificial stimulation. The method may comprise recording the user's behavioural output with a portable electronic device. The portable electronic device may comprise a handheld device, such as a mobile cellphone (e.g. a smartphone). The portable electronic device may comprise a wearable device or implant (e.g. a smartwatch or the like). The method may comprise recording the user's behavioural output with a plurality of devices.

The method may comprise comparing the user's behavioural output with predetermined behavioural outputs. The predetermined behavioural outputs may comprise known behavioural outputs, such as historically determined. The predetermined behavioural outputs may be compiled in a database. The predetermined behavioural outputs may be based upon historical behavioural outputs of the user. For example, the predetermined behavioural outputs may comprise previously-recorded or observed behavioural outputs of the user. Additionally, or alternatively, the predetermined behavioural outputs may comprise previously-recorded or observed behavioural outputs of other users. The predetermined behavioural outputs may be predetermined in advance of the performance of the method of monitoring the user. In at least some examples, the method may comprise a pre-monitoring step or process. The pre-monitoring step or process may comprise compiling the predetermined behavioural outputs, such as in the database. Compiling the predetermined behavioural outputs may comprise observing and/or recording a plurality of behaviours of the user, and optionally other users; such as with the device, or plurality of devices, in loco and/or in a laboratory.

The predetermined behavioural output may be recorded or observed with the portable electronic device, such as the same portable electronic device as used to record the user's behavioural output in the present methods of monitoring a user's neuronal activity. Additionally, or alternatively, the predetermined behavioural output may be recorded or observed with a different device. For example, the predetermined behavioural output may be previously recorded with the same portable electronic device and additionally recorded, such as in a laboratory, with a further behavioural output recording device such as a camera for observing the user. In other examples, the different device may comprise another user's portable electronic device, such as where at least some of the predetermined behavioural outputs are based upon another user's predetermined behavioural outputs.

The behavioural output may be associated with an event-related neuronal activation. For example, a behavioural output of a device interaction, such as a touchscreen touch, may be associated with a particular neuronal activation (e.g. the event-related neuronal activation may comprise a neuronal activation associated with a particular motor control to cause the touch). Each of the behavioural outputs may be associated with respective event-related neuronal activations. The event-related neuronal activations may comprise known event-related neuronal activations. The event-related neuronal activation may comprise an event-based neuronal activation. The event-related neuronal activation may be associated with a provision of an input to or towards the electronic device. The input may comprise one or more of: a gesture, a touch, a sound input, such as voice command, a sequence, a series. For example, the input may comprise a particular sequence of gestures and/or touches. The touch may comprise an ‘air touch’, whereby there is no actual physical contact between user and interface, such as whereby movement of the user's finger towards the device is terminated, withdrawn or redirected prior to contact. Such ‘touches’, or gestures, may be recorded or observed by the portable electronic device (e.g. a proximity sensor/s and/or camera/s, such as of a smartphone). The behavioural output may be indicated by the input to or towards the electronic device. The behavioural output may comprise one or more of: a tap/s; a swipe/s; a gesture/s; a button press; a touchscreen touch; an air touch; a touch; a sound input; a voice command; a sequence; a series; and/or another self paced motor control output/s.

The method may comprise determining the event-related neuronal activation based upon the observed or recorded behavioural output. The method may comprise determining the event-related neuronal activation based solely upon the observed or recorded behavioural output. The method may comprise determining the event-related neuronal activation in dependence on the behavioural output. The method may comprise determining the event-related neuronal activation without observing or recording, such as directly recording or observing, neuronal activity. The method may comprise determining the event-related neuronal activation without a neuronal recorder. The method may comprise determining the event-related neuronal activation without synchronising behavioural recordal or observation with neuronal recording. The method may comprise determining the event-related neuronal activation without synchronising all behavioural recordals or observations with neuronal recordings. The method may comprise determining the event-related neuronal activation outside of a laboratory. The method may comprise determining the event-related neuronal activation in loco. The method may comprise determining the event-related neuronal activation without synchronising, such as without clock synchronising, the behavioural output recorder and the neuronal recorder. The method may comprise determining the event-related neuronal activation in dependence on an unprompted, non-artificial activity or stimuli of the user, such as a normal, day-to-day activity of the user.

The method may comprise an asynchronous correlation between behavioural output and event-related neuronal activation. The method may comprise a non-contemporaneous determination of event-related neuronal activation based upon the observed or recorded behavioural output. The method may comprise the derivation of the event-related neuronal activation in dependence on the behavioural output. The method may comprise matching or identifying the behavioural output with a non-contemporaneous observed or recorded event-related neuronal activation. For example, the method may comprise identifying the behavioural output and associating the behavioural output with a previously-recorded or observed event-related neuronal activation. The method may comprise categorising the behavioural output. The method may comprise determining the associated event-related neuronal activation in dependence on categorisation of the behavioural output.

The method may comprise compiling a database of a plurality of neuronal activities and corresponding behavioural outputs. The method may comprise compiling a database of event-related neuronal activations, such as a database of previously-recorded or observed event-related neuronal activations. The method may comprise compiling the database by matching data from a behavioural output recorder and a neuronal recorder. The data matching may comprise pattern matching to identify event-related neuronal activations, the events being associated with recorded behavioural outputs. In at least some examples, the pattern matching comprises synchronised time-based pattern matching. For example, the database compilation may include synchronous, synchronised neuronal and behavioural output recordings. Accordingly, an event may be identified from a behavioural output recording and a corresponding neuronal activation identified based at least partially upon identification of neuronal activity at a corresponding recorded time or within a corresponding time window or interval. For example, the neuronal activity associated with the behavioural output may be instigated or identified as being initiated in advance of the behavioural output, such as by a time interval associated with a lag or delay between neuronal activity to instigate motor control and the behavioural output caused by the motor control. Additionally, or alternatively, the database compilation may comprise asynchronous pattern matching. For example, the pattern matching may comprise identification of sequences or patterns of behavioural outputs and matching those sequences or patterns with corresponding sequences or patterns of neuronal activity whereby a commonality of absolute or relative time between neuronal and behavioural recordings is not required.

Accordingly, a means, such as the database, for correlating behavioural output with neuronal activity, or vice versa, may be provided. The database may provide a plurality of identifiable event-related neuronal activations. Subsequently the database may be utilised to identify one of neuronal activity or behavioural output based upon the other of behavioural output or neuronal activity. For example, subsequently using only a behavioural output recorder, the associated neuronal activity may be identified based upon matching a pattern from the behavioural output recorded with a pattern stored in the database. Accordingly, the neuronal activity associated with the behavioural output may be identified such as to provide an indication of event-related neuronal activation.

It will be appreciated that the database may be supplemented or adapted subsequent to its establishment. For example, additional data or inputs may be utilised to identify additional event-related neuronal activations. Similarly, the database may be updated to reflect an identified deviation or adaptation of patterns, such as over time and/or with different or additional users and/or behavioural outputs.

The method may comprise comparing the user's behavioural output with an event-related neuronal activation matching a pattern associated with a behavioural output recorder with known patterns to identify an event-related neuronal activation. The known patterns may have been previously established using a neuronal recorder. The method may comprise determining the user's event-related neuronal activation based upon the comparison of the user's behavioural output with predetermined behavioural outputs, such as stored in the database. The method may comprise determining the user's event-related neuronal activation based upon the comparison so as to provide an indication of the user's neuronal activity. The method may comprise identifying a development in the user based at least predominantly on monitoring only via the behavioural output recorder in the form of the portable electronic device.

The method may comprise recording a plurality of behavioural outputs of the user; and using the plurality of behavioural outputs to determine the neuronal activity of the user. The plurality of behavioural outputs may be sequential, over a period of time. The plurality of behavioural outputs may be recorded by a same, single behavioural output recorder, such as a smartphone. Optionally, the plurality of behavioural outputs may be recorded by a plurality of devices, such as a user's smartphone and the user's tablet or laptop.

The method may comprise determining the user's neuronal activity over a period of time, sequentially. The method may comprise determining a/any change in the user's neuronal activity over the period of time. The change in the user's neuronal activity may be associated with a development of the user. For example, the development may be associated with an improvement or deterioration in the neural activity of the user. The development may be associated with a health of the user. The method may comprise associating the user's neuronal activity with one or more of: physical wellbeing; mental wellbeing; physical development/s; mental development/s; treatment; disease; diagnosis. For example, the method may comprise identifying a development in a particular region or area of the brain, based at least predominantly on only the recorded behavioural output. For example, a change in identified event-related neuronal activations over a period of time may be associated or associatable with a particular function or area of the brain. Accordingly, the change may be associated or associatable with a corresponding change in the function and/or area of the brain. The change may be associated with an impairment or disease and/or a treatment thereof. For example, the method may comprise a diagnosis, particularly early diagnosis, of an ailment associated with a particular function or area of the brain. For example, the method may comprise identifying or diagnosing a development and/or treatment of a disease or ailment, such as one or more of: a brain injury; a brain disease; cancer; tumour; Parkinson's, Multiple Sclerosis; dementia; cerebral palsy; stroke; epilepsy. In at least some examples, the change in identified event-related neuronal activations over a period of time is indicative of a particular change in function or condition of a particular area of the brain, such as identified in the illustrated examples (e.g. in the contralateral sensorimotor cortex). The method may comprise alerting the user and/or a third party, such as a medical professional; as to the development or change. The alert may comprise a realtime alert, such as an emergency alert. Additionally, or alternatively, the method may comprise monitoring effects on neuronal activity, such as associated with user-based activities; medications; recreational activities or drugs; or the like. Additionally, or alternatively, the method may comprise monitoring the user's behaviour and/or development, such as socially.

In at least one example, a method of the present disclosure enables a generation of event-related analysis of the neuronal data by empirically aligning the two data streams of a person's known taps and the persons' continuously recorded brain signals, such as using the method of FIG. 1, by leveraging the known features of SmRP. It will be appreciated, that in at least some examples, once the signals are aligned the data can be processed in: (a) time-voltage space: x-axis time, y-axis voltage; and/or (b) frequency-power space: x-axis frequency of the brain signal, y-axis power; and/or (c) other parameters. It will be appreciated that although the alignment is shown here with signal of the form ‘a’, the subsequent analysis may be in any dimension (e.g. ‘a’, ‘b’ and/or ‘c’).

According to a further aspect there is provided a method of simulating or modelling the method and/or apparatus according to any other aspect, embodiment, example or claim.

Another aspect of the present disclosure provides a computer program comprising instructions arranged, when executed, to implement a method in accordance with any other aspect, example, claim or embodiment. A further aspect provides machine-readable storage storing such a program. The storage may be non-transitory.

According to an aspect of the invention, there is provided computer software which, when executed by a processing means, is arranged to perform a method according to any other aspect, example, claim or embodiment. The computer software may be stored on a computer readable medium. The computer software may be tangibly stored on a computer readable medium. The computer readable medium may be non-transitory. The computer software may comprise a smartphone application, such as a background App.

According to an example of the present disclosure there is provided a method of analyzing a functioning of neuronal circuits of a brain of an individual. Here, the circuits are engaged in the individual's voluntary, self-paced motor control of a button press with a finger, Here, the method comprises: measuring the smartphone related potential (“SmRP”) of the brain of the individual when the individual uses, particularly with the individual's thumb, a touch screen of a smartphone; and then comparing the measured SmRP of the brain of the individual with standard measured values of SmRP of brains of other individuals when the other individuals use, particularly with the other individuals's thumbs, touch screens of smartphones. Such measurements may be utilised to compile a database of neuronal activities corresponding to behavioural outputs.

According to an example of the present disclosure, there is provided a system for analyzing a functioning of neuronal circuits of a brain of an individual, which circuits are engaged in the individual's voluntary, self-paced motor control of a button press with a finger, the system comprising: a smartphone with a touch screen; an apparatus for scanning the brain of the individual to measure the SmRP of the brain of the individual when the individual uses, particularly with the individual's thumb, the touch screen of the smartphone; and means for comparing the measured SmRP of the brain of the individual with standard measured values of SmRP of brains of other individuals when the other individuals use, particularly with the other individual's thumbs, touch screens of smartphones.

According to an example of the present disclosure, there is provided a use of a smartphone for analyzing a functioning of neuronal circuits of a brain of an individual, which circuits are engaged in the individual's voluntary, self-paced motor control of a button press with a finger, the use comprising; determining the SmRP of the brain of the individual when the individual uses, particularly with the individual's thumb, a touch screen of a smartphone; and comparing the determined SmRP of the brain of the individual with standard determined values of SmRP of brains of other individuals when the other individuals use, particularly with the other individuals's thumbs, touch screens of smartphones.

The invention includes one or more corresponding aspects, embodiments, examples or features in isolation or in various combinations whether or not specifically stated (including claimed) in that combination or in isolation. For example, it will readily be appreciated that features recited as optional with respect to the first aspect may be additionally applicable with respect to the other aspects without the need to explicitly and unnecessarily list those various combinations and permutations here (e.g. the method of one aspect may comprise features of any other aspect). Optional features as recited in respect of a method may be additionally applicable to an apparatus or device; and vice versa. The apparatus or device of one aspect, example, embodiment or claim may be configured to perform a feature of a method of any aspect, example, embodiment or claim. In addition, corresponding means for performing one or more of the discussed functions are also within the present disclosure.

It will be appreciated that one or more embodiments/aspects may be useful in at least monitoring a user.

The above summary is intended to be merely exemplary and non-limiting.

Various respective aspects and features of the present disclosure are defined in the appended claims.

It may be an aim of certain embodiments of the present disclosure to solve, mitigate or obviate, at least partly, at least one of the problems and/or disadvantages associated with the prior art, such as described herein or elsewhere. Certain embodiments or examples may aim to provide at least one of the advantages described herein.

### BRIEF DESCRIPTION OF THE DRAWINGS

FIG. 1 shows an example of a method of compiling a database, with event-related neuronal activations.

FIG. 2 shows an example of a method of monitoring a user's neuronal activity, using a behavioural output recorder without requiring a neuronal recorder.

FIGS. 3A-3E show the SmRP of touchscreen events with the rapid engagement of distinct cortical processes surrounding the events. (FIG. 3A) Shows SmRP isolated by aligning the touchscreen events while users used their own smartphones with the recorded EEG signals. (FIG. 3B) Shows the time-course of the population of grand average of signals detected over the scalp from select electrodes and the corresponding standard error of the mean. (FIG. 3C) Shows the topology of grand average signals detected over the scalp. (FIG. 3D) Shows the corresponding results of one-sample t-tests. (FIG. 3E) Shows the latency to the statistically significant signal onsets. (FIG. 3F) Shows the offsets to the statistically significant signal onsets. FIGS. 4A-4C show how the signals over the sensorimotor cortex are depressed when

on social apps vs. non-social apps. (FIG. 4A) Shows population grand average of the kinematic profiles of the thumb movements used towards social and non-social Apps. The shaded area depicts the standard error of the mean. (FIG. 4B) Show the time-course of the population average of the regression-adjusted—for trial-to-trial kinematic variation—EEG signals. Insert shows the un-adjusted signals; the shaded are depicted the standard error. (FIG. 4C) Shows the topology of population means of adjusted signals and the outcomes of the paired t-test comparing SmRP gathered from the social vs. non-social Apps.

FIGS. 5A-5C show how the signals over the sensorimotor cortex are over-turned for ‘air touches’ vs. real touchscreen touches. (FIG. 5A) Shows population grand average of the kinematic profiles of the thumb movements during ‘air touches’, i.e., when the thumb flexion occurs without a touchscreen event, and real touchscreen events. (FIG. 5B) Shows the time course of the population average EEG signals recorded over the sensorimotor cortex. (FIG. 5C) Shows the topology of the grand average signals detected over the scalp for the air touches vs. touchscreen touches, and the outcomes of the paired t-test comparing the two event types.

### DETAILED DESCRIPTION

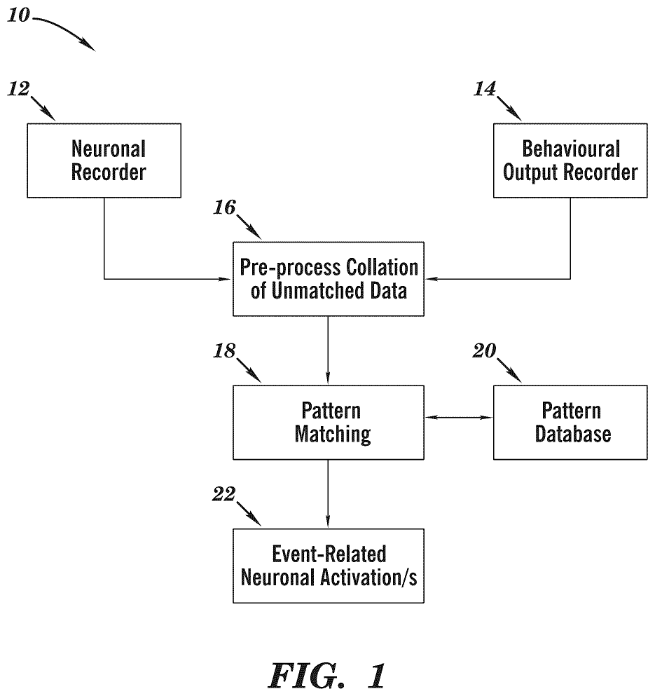

FIG. 1 illustrates an example of a method 10 according to the present disclosure. The method 10 shown here comprises compiling a database 20 of a plurality of neuronal activities and corresponding behavioural outputs. The method 10 comprises compiling a database of event-related neuronal activations 22, here being a database of previously-recorded or observed event-related neuronal activations. The method comprises a pre-process collation of unmatched data 16 from a neuronal recorder 12 and a behavioural output recorder 14. The method 10 comprises compiling the database 20 by matching 18 data from the behavioural output recorder 14 and the neuronal recorder 12. The data matching comprises pattern matching 18 to identify event-related neuronal activations 22, the events being associated with recorded behavioural outputs. In at least some examples, the pattern matching 18 comprises synchronised time-based pattern matching. For example, the database 20 compilation includes synchronous, synchronised neuronal and behavioural output recordings. Accordingly, an event 22 is identified from a behavioural output recording 14 and a corresponding neuronal activation identified based at least partially upon identification of recorded neuronal activity at a corresponding recorded time or within a corresponding time window or interval. For example, the neuronal activity associated with the behavioural output is often instigated or identified as being initiated in advance of the behavioural output, such as by a time interval associated with a lag or delay between recorded neuronal activity 12 to instigate motor control and the recorded behavioural output 14 caused by the motor control. It will be appreciated that the method 10 of FIG. 1 can be supplemented or improved, such as with subsequent iterations or steps to expand and/or refine the database 20. For example, the patterns identified herein can be further improved with increasing data. More details in the patterns identified herein may emerge as the data collection pipeline improves. For instance, a small peak that can occur at roughly 50 ms after a touch (capturing the touch-related brain activity) may not show up with the presently-illustrated method (e.g. present resolution), but can be included in the method 10 and database 20 subsequently (e.g. with increased resolution or refinement).

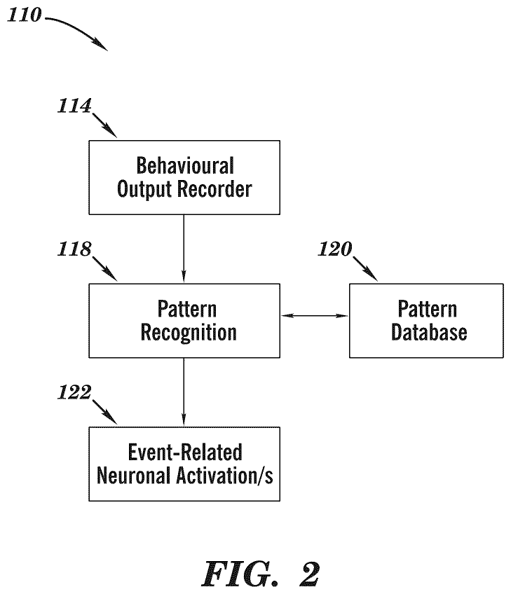

FIG. 2 illustrates an example of a method 110 according to the present disclosure. The method is provided for monitoring a user's neuronal activity. The method comprises recording a user's behavioural output 114 with a behavioural output recorder, typically a portable electronic device, such as a mobile cellphone. The method 110 comprises comparing the user's behavioural output with predefined behavioural outputs associated with known event-related neuronal activations. As shown here, the method comprises utilising pattern recognition 118 of the behavioural output and pattern matching with the pattern database 120 to determine 122 the user's event-related neuronal activation based upon the comparison so as to provide an indication of the user's neuronal activity. Accordingly, the neuronal activation 122 is effectively modelled in the method of FIG. 2, being inferred or derived without direct measurement of neuronal activity with a neuronal recorder as such.

In at least some examples, the method comprises recording a plurality of behavioural outputs of the user; and using the plurality of behavioural outputs to determine the neuronal activity of the user. The method comprises determining the user's neuronal activity over a period of time, sequentially, to identify a development in the user's neuronal activity. The method comprises associating the user's neuronal activity with one or more of: physical wellbeing; mental wellbeing; physical development/s; mental development/s; treatment; disease; diagnosis. The method comprises comparing the user's behavioural output with an event-related neuronal activation matching a pattern associated with a behavioural output recorder with known patterns to identify an event-related neuronal activation; and the known patterns are previously established using a neuronal recorder. The method comprises comprises compiling a database of a plurality of neuronal activities and corresponding behavioural outputs. It will be appreciated that the method comprises compiling the database in advance of performing the monitoring of the user's neuronal activity, such as with the method as shown in FIG. 1. T will be appreciated that the database 120 shown in FIG. 2 may be the same as the database 20 developed or established in the method of FIG. 1. The method comprises pattern matching of behavioural output with neuronal activity to enable identification of one of behavioural output or neuronal activity based on only one of the other of neuronal activity or behavioural output. The database 120 enables the identification of neuronal activity based solely on recording or observing behavioural output, without requiring direct neuronal recording. Here, the method of FIG. 2 does not comprise synchronising the behavioural output recorder and the neuronal activity recorder. The method comprises the asynchronous recording of behavioural output and neuronal activity.

As will be described in more detail below, the event-related neuronal activation is associated with a provision of an input towards the portable electronic device. The input comprises one or more of: a gesture; a touch; a sound input, such as voice command; a sequence; a series. In at least some examples, the method comprises a method of diagnosis, the user comprising a patient. Similarly, in at least some examples (potentially overlapping examples), the monitoring comprises assessing the user's cognitive function. For example, cognitive tasks may involve the processing of salient information regardless of the modality used for the inputs. The MP of an individual may be depressed by an increased cognitive load with no overt motor impact. Emotionally laden stimuli may depress MP related signals. Accordingly, assessment of the behavioural ouput (via the input to the device) may provide indications of the user's cognitive function.

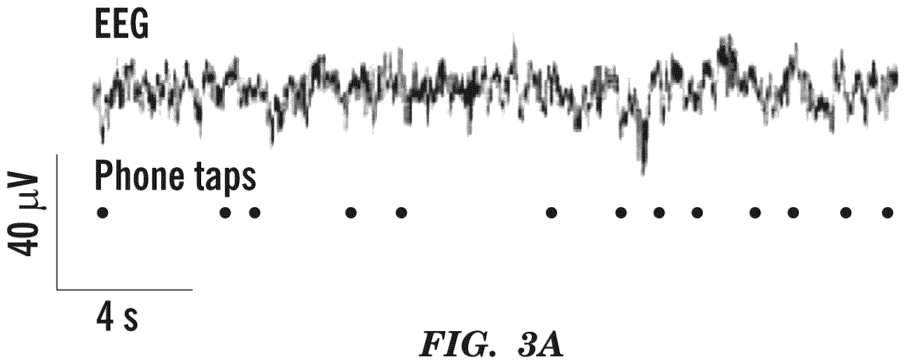

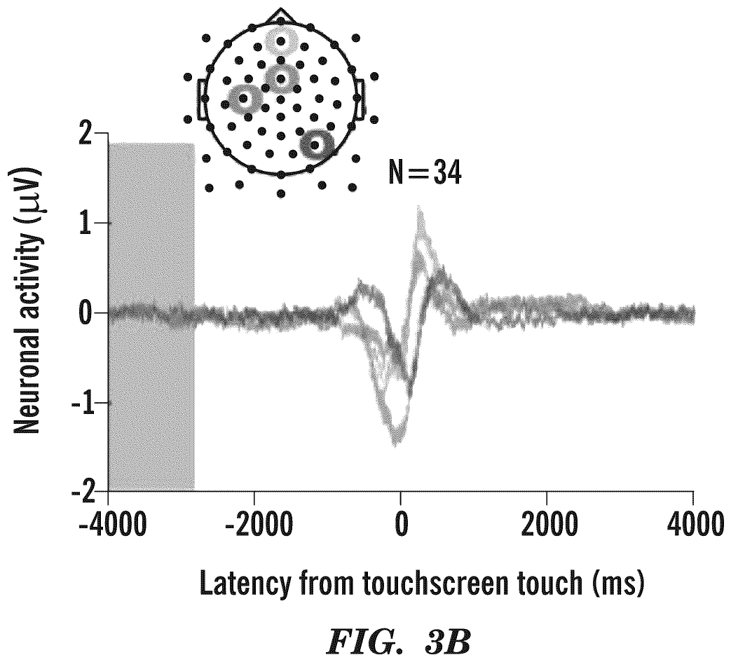

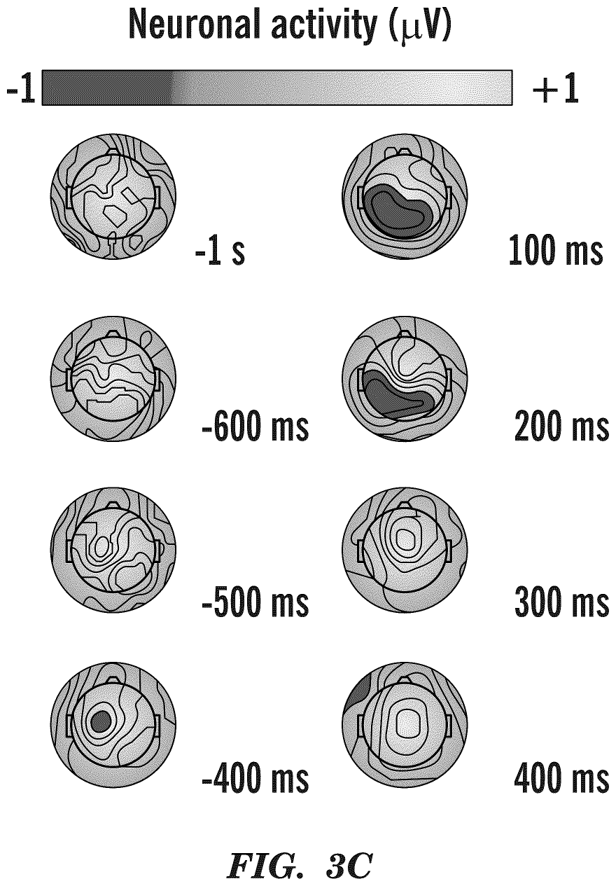

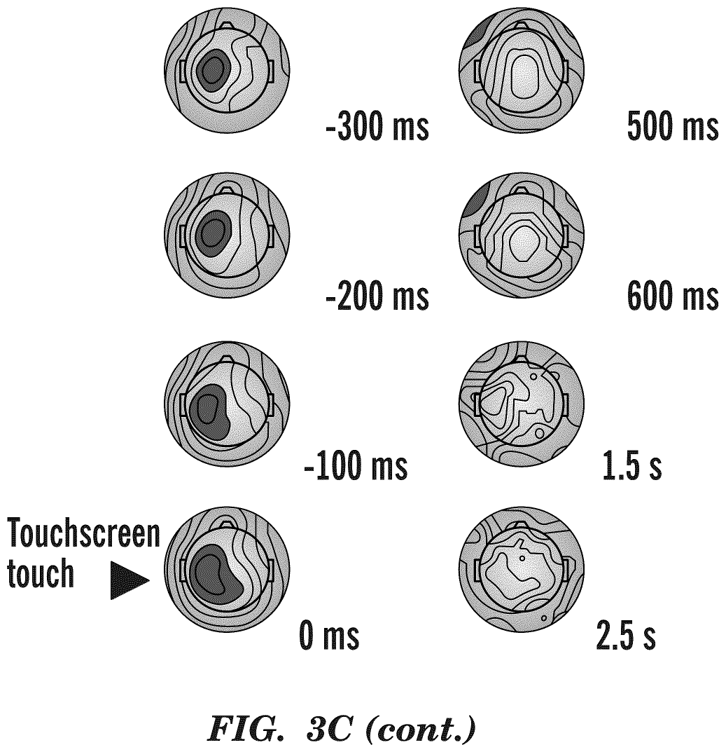

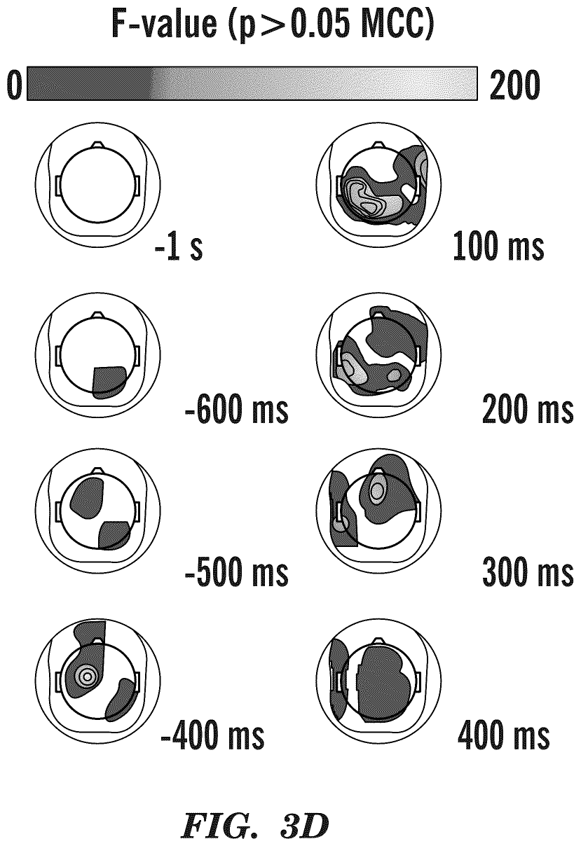

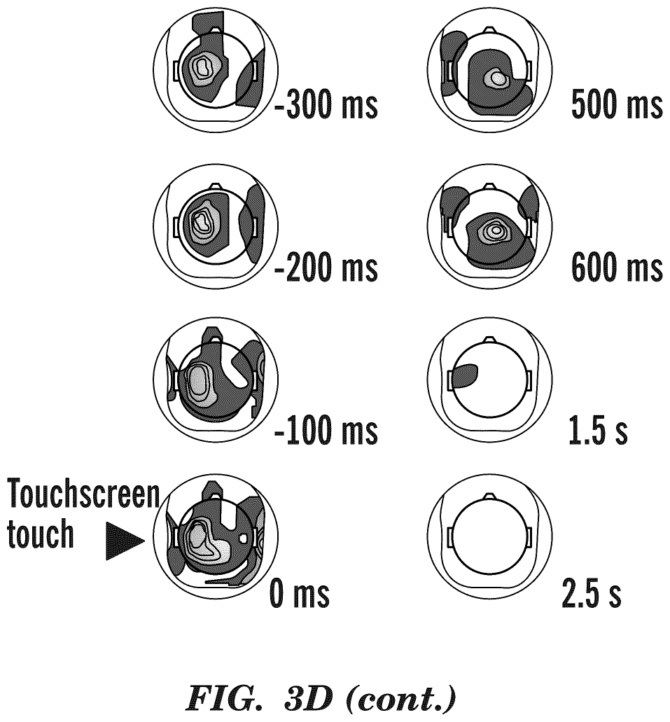

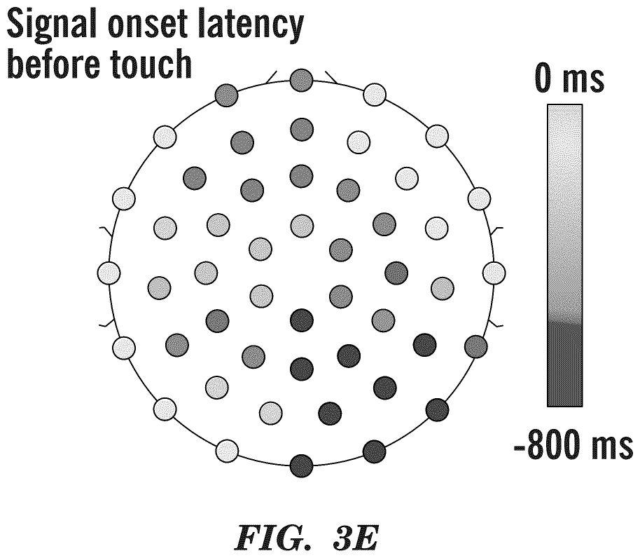

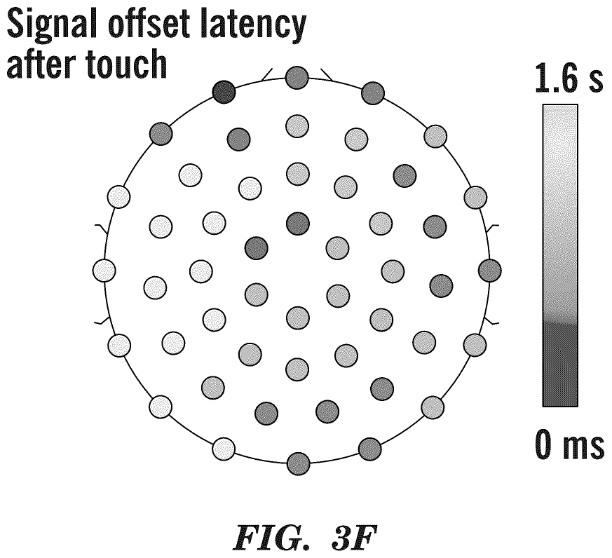

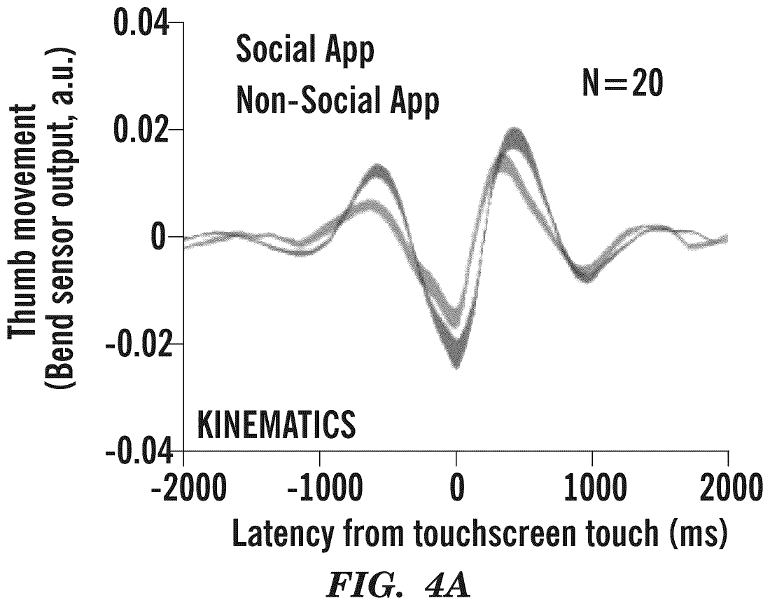

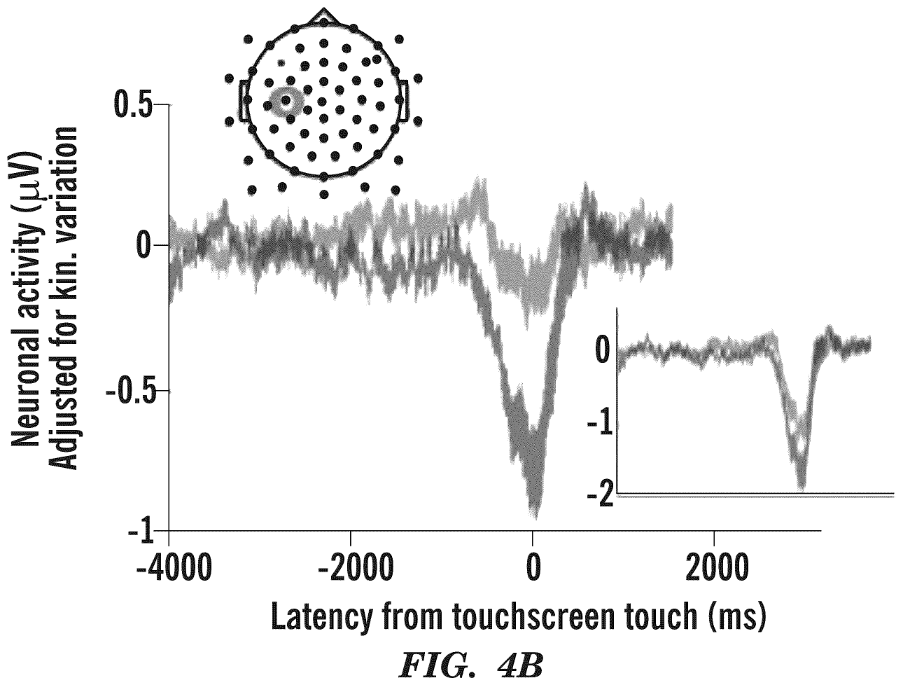

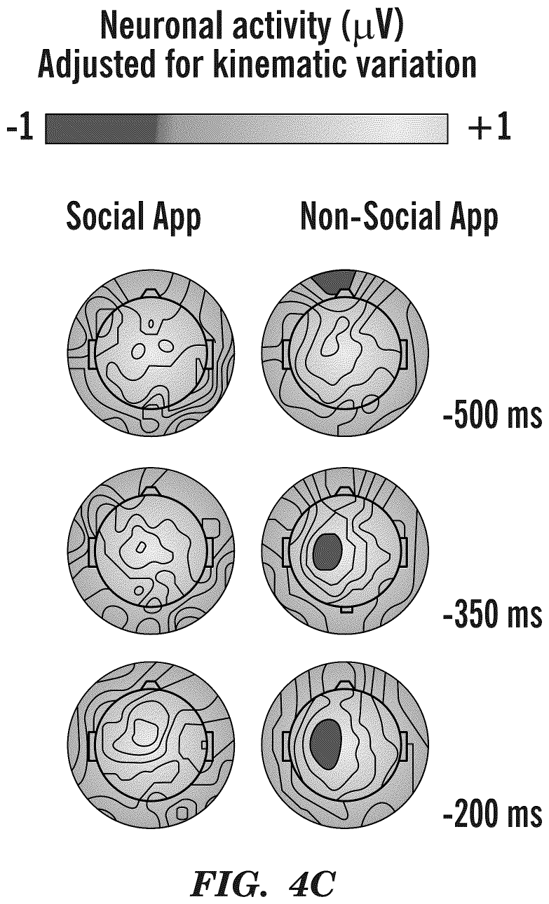

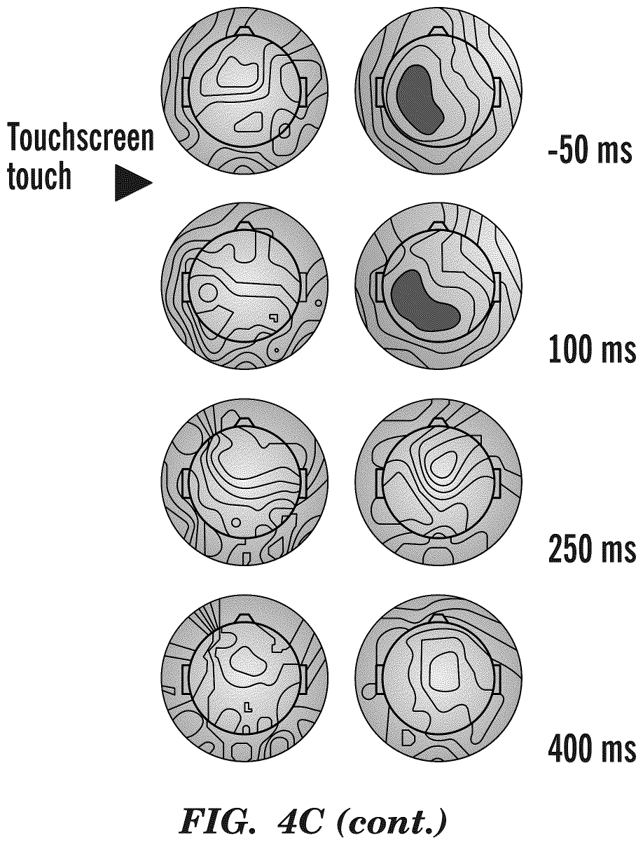

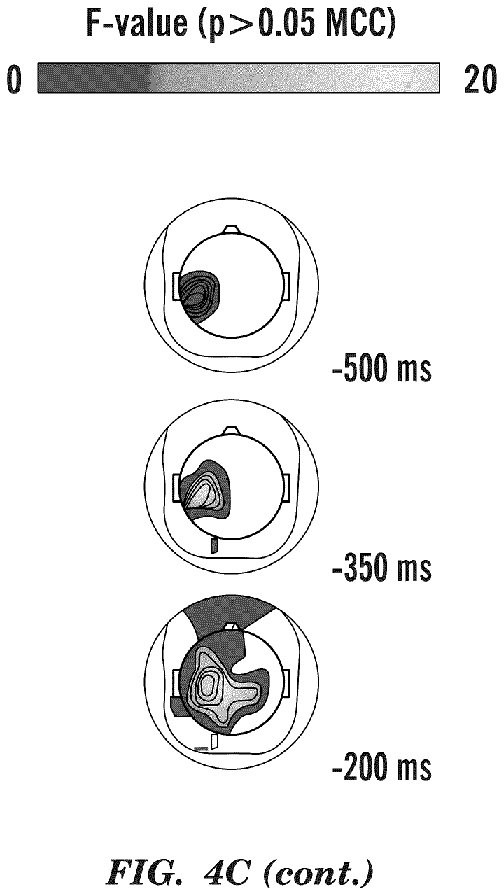

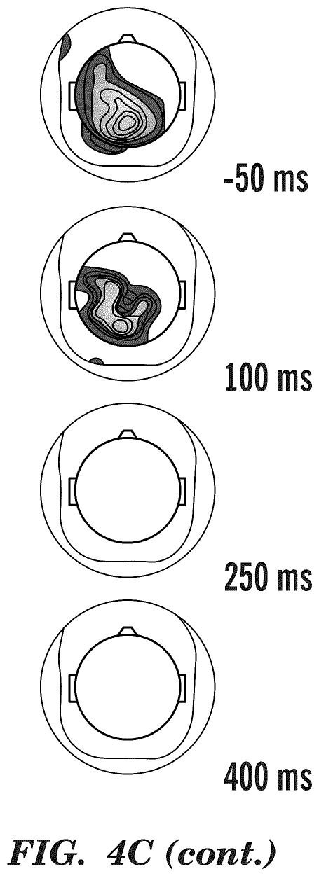

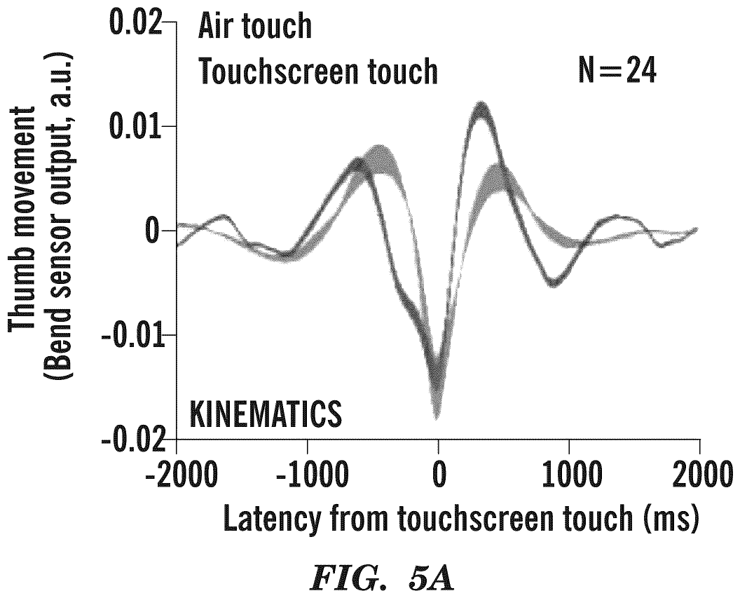

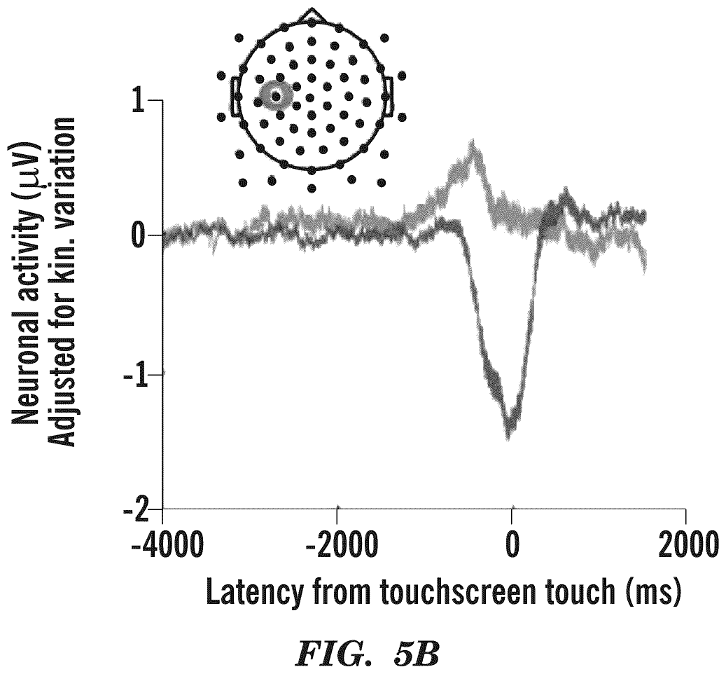

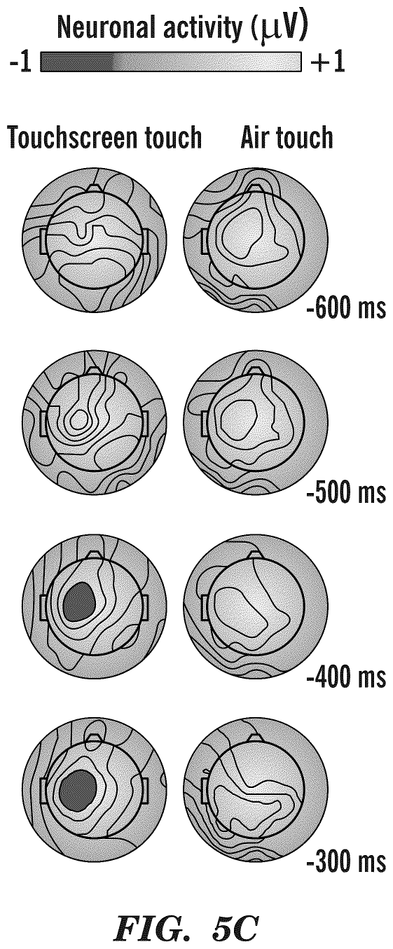

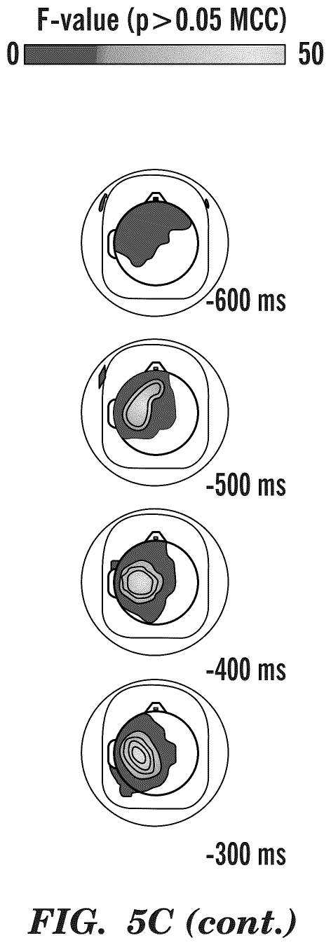

FIG. 3 illustrates a method of analyzing a functioning of neuronal circuits of a brain of an individual, which circuits are engaged in the individual's voluntary, self-paced motor control of a button press with a finger, the method comprising: measuring the smartphone related potential “SmRP” of the brain of the individual when the individual uses, particularly with the individual's thumb, a touch screen of a smartphone; and then comparing the measured SmRP of the brain of the individual with standard measured values of SmRP of brains of other individuals when the other individuals use, particularly with the other individual's thumbs, touch screens of smartphones. In “a” of FIG. 3, a series of sequential ‘phone taps’ is shown (as dots) over a time interval, along with the corresponding EEG readings (in μ).

As used herein, the term “smartphone related potential” or SmRP preferably means one or more, preferably all, of the following: the readiness potential (“RP”), the motor potential (“MP”), the reafferent potential (“RAP”) of the brain of an individual, the consecutive post movement sensory processing involving the tactile, visual, frontal & parietal electrodes.

The method involves comparing the smartphone related potential (“SmRP”) of an individual's touchscreen events with the rapid engagement of distinct cortical processes of the individual surrounding the events.

Initially, SmRP is measured prior to and following any touchscreen event by the individual. For this purpose, EEG signals of the individual are measured while the individual is engaged in spontaneous right-handed (thumb) touchscreen touches on his/her own smartphone to reveal the neuronal activity surrounding the touchscreen event. The population median of inter-touch intervals of the analyzed events can be 2 s (a 700 ms inter-touch interval cutoff can be used to eliminate the fast touchscreen events). It is estimated that the EEG signal population average to capture the statistically significant deviations from a 1 s long baseline starting at 4 s prior to the touch. A flat recording can persist for up to 704 ms prior to the touch and the earliest signal can be detected at the right parietal and occipital electrodes (FIG. 3). According to the population average, this posterior positive signal can be briefly followed by the simultaneous activation of the frontal (negative) and the parietal & occipital (positive) electrodes. The gap seen at signal onsets between the posterior and anterior electrodes can also be apparent at the corresponding signal peaks. By 400 ms prior to the touch, the negative signals over the contralateral (left) sensorimotor cortex can dominate the topology. At the time of the touchscreen event (0 ms from the event), the negative signals can additionally occupy the parietal and occipital electrodes bilaterally.

Then, SmRP is measured after a touchscreen event by the individual. With the touchscreen event, the signals over the sensorimotor cortex can begin to reverse from the negativity. The bilateral negative components over the parietal and occipital electrodes, which can develop prior to the touch, can peak in the first 100 ms after the touch (FIG. 3). In the grand average signal, the negative peak latency can be the shortest over the sensorimotor cortex followed by the frontal electrodes and then the parietal electrodes. In the subsequent 200 ms, these negative components can be entirely replaced by a distinct positive component occupying the central and the frontal electrodes. By 400 ms after the touchscreen event, the positive component can occupy the central and parietal electrodes. This propagation towards the posterior electrodes can continue with activation over the parietal and occipital electrodes at 600 ms. This pattern of sequential activation from the frontal-to-occipital electrodes can also be apparent in the latency to the signal peaks. Although this wave of activation can subside by 700 ms, the signals over the left sensorimotor cortex can remain higher than the baseline until 1995 ms after the touchscreen event.

The variations in the amplitude of the negative sensorimotor signal detected before the touch between different individuals shows that the pre-touch negativity can be correlated with the post-touch activity. The pre-touch activity can be correlated almost exclusively over the parietal & occipital electrodes. The higher the amplitude of pre-touch activity the larger is the positive component over the parietal and occipital electrodes between 200-600 ms.

The effect on pre-touch neuronal activity of an individual between social and non-social Apps is also measured. Apart from measuring the brain signals of the individual, the thumb flexion and the extension of the thumb of the individual are also preferably measured, preferably by using bend sensor recordings (see e.g. “a” of FIGS. 4 and 5). Kinematically, the amplitudes of the thumb movements are generally similar but with a tendency for the movements being of higher amplitudes when using non-social Apps compared to social Apps (the differences are not generally statistically significant after multiple comparison correction (FIG. 4a). For either category, the touchscreen events generally start with a brief thumb extension and a descent towards the screen (flexion) at ˜600 ms prior to the touch (based on the population mean, 619 ms for social Apps and 571 ms for non-social Apps). After the touch, the thumb is generally more rapidly withdrawn from the screen than the descent towards the screen reaching the maximum flexion already at ˜400 ms (based on the population mean, 341 ms for social Apps and 426 ms for non-social Apps). The SmRPs of individuals are found to differ according to the behavioral context. In this regard, the pre-touch SmRPs over the sensorimotor cortex are depressed when engaged in social vs. non-social Apps in terms of signal amplitude (FIG. 4c). The reduced signal amplitude is apparent at 500 ms before the touchscreen event. The differences mainly occur in the electrodes over the sensorimotor cortex, but the negative components engaging the left parietal and occipital electrodes are also depressed, and this depression can last for up to 100 ms after the touch. The depressed sensorimotor negativity is also present in the analysis of the kinematically unadjusted potentials.

The effects of ‘air touches’ on SmRP is also measured. In this regard, while individuals are using their smartphone, their thumbs generally are at times flexed towards the screen without resulting in any touchscreen event (FIG. 5a). These ‘air touches’ account for a mean of 31.66% (±3.0% SE) of all the thumb flexions towards the screen. Indeed, ‘air touches’ have been found to be inversely proportional to the number of real touchscreen events (β=−0.0004, R2=0.667, p=2.01×10−07, t=−7, linear regression analysis). As real touchscreen events occur at maximum thumb flexions, EEG analysis can be correlated to the maximum flexions. Both the “air touches” and the real touchscreen events are then seen to share similar movement profiles, starting with a thumb extension and then a flexion towards the screen (at 437 ms before the air touch, based on the population mean) followed by withdrawal (extension) away from the screen. However, the final extension for the “air touches” is not as extensive as for the real touchscreen touches.

The SmRP prior to an “air touch” is also compared to the SmRP prior to an actual touch, starting from 680 ms prior to the touch (FIG. 5b & c). Significantly, an actual touch yields a strong pre-touch negative component over the sensorimotor cortex while an air touch yields a positive component over the sensorimotor cortex. The positive component peaks at 487 ms (based on the population mean) over the sensorimotor electrodes before the air touch.

The method of measuring the different SmRP potentials, i.e., the RP, the MP and the RAP associated with a touchscreen event involving a smartphone shows that a series of neuronal activations are generally involved. In this regard, a touch is preceded by posterior-to-anterior EEG signal flow and strong activation of the sensorimotor cortex. It is followed by an opposite anterior-to-posterior signal flow, unraveling the distinct directions of cortical information flow associated with touching the screen and processing the consequences of the touch respectively. The activation of the sensorimotor cortex is strongly modulated by the behavioral context in terms of the App in use and the near-term consequences of the thumb movements (as in if a movement was followed by touch or not).

It has been found, by this method, that touchscreen movements by an individual are rapidly prepared and that the crucial decision by the individual to touch or not to touch the screen of a smartphone can occur with movement initiation. The first consistently visible signals before the touchscreen event are detected over the frontal and parietal (and occipital) electrodes about 700 ms before the touchscreen event, while the thumb was already extended to descend towards the screen at ˜600 ms before the touch. Such frontal-parietal signals seen prior to dominant negativity over the sensorimotor cortex are associated with visuomotor attention and response selection. This suggests that neuronal activity precedes the movements by only 100 ms-20× faster than the 2 s preparatory time observed in slow laboratory finger tapping tasks. However, an extended thumb does not always lead to a touchscreen event and such an event is accompanied with a distinct positive component over the sensorimotor cortex starting at ˜700 ms prior to the ‘air touch’. Therefore, the decision process underlying a touchscreen event and the motor control processes can be highly compressed in time on the smartphone. Although screen touches separated by 2 s are common, more rapid are frequent, separated by less than 500 ms (median).

It has also been found that the pre-touch negativity over the sensorimotor cortex is depressed when engaged in social Apps compared to the non-social Apps. This difference is apparent from ˜500 ms prior to the touch up to ˜100 ms after the touch, even when any contribution of motor amplitude fluctuations are regressed out at the level of individual trials. This suggests that the sensorimotor computations remain connected to the behavioural context through much of the ongoing action.

The SmRP potentials, measured by this method, suggest the following. Firstly, a touch is followed by continued negativity over the contralateral sensorimotor cortex and enlargement of the negativity bilaterally to occupy the parietal and occipital electrodes. These signals most likely reflect action monitoring and tactile-visual confirmation of the touchscreen event. Secondly, there is a positive component that sequentially recruited the anterior to the posterior electrodes. This pattern of activity and the signal latency is consistent with the P3 (P300) component which reflects the attention and memory-related brain. This wave is commonly observed in cognitive tasks involving the processing of salient information regardless of the modality used for the inputs. The neuronal generators underlying this signal likely inhibit extraneous information flow and thus enhance the flow of information from an attention-grabbing input from the frontal to the parietal cortical structures to ‘sharpen memory. The post-touch cognitive processing may, in fact, be shaped by the pre-touch sensorimotor activity, as the amplitude of the sensorimotor signals recorded prior to the touch was correlated to the post-touch activity over the parietal and occipital electrodes.

Accordingly, it can be seen that patterns matching behavioural output with neuronal activity can be identified. Such patterns can be stored in a database. For example, particular touches associated with particular smartphone functions can be matched to particular neuronal activities. These patterns can then be used, such as in the method of FIG. 2, to identify event-based neuronal activations based purely on the behavioural output. Accordingly, the smartphone can be used as a behavioural output recorder, such as with software (e.g. a background app) recording behavioural output of the user. The behavioural output can be matched, either realtime or subsequently, to patterns in the database to identify the user's neuronal activity. Particularly over a longer period of time, developments in a user's neuronal activity can be identified. For example, temporal changes in a user's event-related neuronal activations may be identified. Such changes may be associated with improvement and/or deterioration of a user. For example, where a user is a patient, such as a neurological patient, changes in neuronal activity over a period of time may be identified based solely, or at least predominantly, on smartphone use—particularly, normal daily unprompted smartphone use by the user in loco. Such changes may be associated or associatable with physical or mental changes, such as of health. Accordingly, the changes may represent an improvement, such as recovery or healing; or the changes may represent a deterioration, such as a medical setback. Accordingly, the behavioural output as monitored by the smartphone app may act as a trigger to take action, such as to request or instigate a consultation or laboratory analsysis or follow-up with the user.

The measured SmRP of the brain of an individual can be compared with standard measured values of SmRPs of brains of other individuals when the other individuals use, particularly with the other individual's thumbs, touch screens of smartphones. From this comparison, one can readily analyse one or more of the individual's wellbeing, health, preferences, predilections, fears, biases, loyalties and the like.

It will be appreciated that the method can be we can include estimates of ‘accuracy’ of alignment, and optionally distinguish outcomes in healthy and diseased individuals. It will also be appreciated that the exact detailed shape of the SmRP may be refined. For example, further peaks and valleys may be revealed or identified as the database 20 grows. In at least some examples, the coarse features as shown here may remain stable but the finer features may be altered.

With over 20% of the global population on smartphones, the touchscreen movements are one of the most common actions and yet one of the simplest in appearance. Understanding how these actions are generated can not only reveal fundamental insights into how the brain engages in complex behavior but also offers a new avenue to measure brain functions relevant to the real world.

Also in accordance with this invention, a system is provided for analyzing a functioning of neuronal circuits of a brain of an individual, which circuits are engaged in the individual's voluntary, self-paced motor control of a button press with a finger, the system comprising: a smartphone with a touch screen; an apparatus for scanning the brain of the individual to measure the SmRP of the brain of the individual when the individual uses, particularly with the individual's thumb, the touch screen of the smartphone; and means for comparing the measured SmRP of the brain of the individual with standard measured values of SmRP of brains of other individuals when the other individuals use, particularly with the other individuals's thumbs, touch screens of smartphones.

Also in accordance with this invention, a use of a smartphone for analyzing a functioning of neuronal circuits of a brain of an individual, which circuits are engaged in the individual's voluntary, self-paced motor control of a button press with a finger, the use comprising; determining the SmRP of the brain of the individual when the individual uses, particularly with the individual's thumb, a touch screen of a smartphone; and comparing the determined SmRP of the brain of the individual with standard determined values of SmRP of brains of other individuals when the other individuals use, particularly with the other individuals's thumbs, touch screens of smartphones.

### EXAMPLE

A total of 45 people were recruited. The sample age ranged from 18 to 45 (median age, 23).

### Smartphone Activity Recordings

The timestamps of the touchscreen interactions and the corresponding Apps in use were recorded by using a background App attached to a cloud-based data collection platform (TapCounter, QuantActions Ltd, Lausanne, Switzerland). The background App was installed for a period of 3-5 weeks prior to the laboratory-based EEG recordings. This data were downloaded from the cloud in a compressed form and further processed using MATLAB (Mathworks, Natick, USA) by using the data unpacking processes made available by QuantActions.

### Categorization of Social and Non-Social Apps

The classification of social and non-social Apps was primarily based on the definitions used in a previous report. To elaborate, an app label captured on the phone was categorized as social if the main purpose was to allow users to communicate with others (friends or strangers). The app had to also contain the tools to enable these interactions such as direct messaging, public posts or voice chat, personalized profiles and being able to rate or follow the profile. The apps that did not fit the main purpose and have the tools were labeled as non-social. A slightly altered definition was applied when categorizing gaming apps—they were categorized as social only if they engaged other users during the game rather than sharing the results after playing the game solo.

### Movement Sensor Recordings

During the laboratory measurements of smartphone behavior, the thumb flexions were tracked using a bend sensor (Flex Sensor, 4.5″, Spectra Symbol, Salt Lake City, USA). The sensor was attached to the thumb (dorsum) using a custom-built jacket that allowed the sensor to bend within the jacket without the sensor being pulled. The thumb was further covered with a conductive surface (aluminum foil) ensuring that all the touches were translated to touchscreen events and that the same part of the thumb was used to target the screen. The analog signals from the sensor were digitized at 1 kHz using Labview via the USB 6008 DAQ (National Instruments, Austin, USA). The same DAQ was also used to power the sensor. The thumb was all able to freely move on the touchscreen under this configuration. The movements were recorded along with synchronizing triggers emitting from the EEG recording set-up.

### EEG Recordings

The EEG recordings were conducted in a faraday shielded room (Holland Shielding Systems BV, Dordrecht, The Netherlands) with optic fiber transmitted internet connectivity. The users used their own smartphone while comfortably reclined on a chair. White noise was presented through the experiment using earphones. Sixty-four channel EEG caps with equidistant electrodes were used (Easycap GmbH, Wörthsee, Germany) in conjunction with ABRALYT HiCl electrode gel. Prior to recording the EEG signals, the contact impedances were reduced to less than 10 kΩ by rubbing the gel on the skin. The capsizes were matched using head circumference measurements. The EEG recordings were conducted using BrainAmp DC amplifiers (Brainproducts, Gilching, Germany). The data sample rate was set at 1 kHz and no online filters were applied during the recordings. As the EEG, the smartphone and the movement sensors operated on different clocks they were synchronized using common TTL pulse bursts generated by using an IBM T 42 motherboard running MATLAB.

To measure the EEG signals surrounding the smartphone behavior, the users were provided with a list of their own top 4 apps-top 2 social and non-social ranked based on the number of touches generated over the previous weeks. Video Apps such as youtube were eliminated prior to the ranking. The users were given 12 minutes to use each App, and the usage was separated by 2 minute short breaks where the subsequent App was launched and ready to be used. The order of the Apps was randomized. Social interactions were anticipated to be dominated by higher levels of text messaging compared to the non-social interactions. To enable comparison between the social and non-social interactions we instructed the participants to ‘not engage in extensive text messaging’—and this was further monitored by the experimenter by using the thumb flexion bend sensor measures online. However, users were permitted ˜50 characters of typing in situations where they judged that typing was essential for continued engagement (for instance when writing a short reaction to a posted picture). According to debriefing interviews, users mainly browsed older posts, read and liked the posts, and responded with brief comments using emojis and a few characters.

### EEG Analysis and Statistics

The temporal alignment between the smartphone touchscreen events and the bend sensor recordings was confirmed using cross-correlation analysis (Signal processing toolbox, MATLAB). In 8 of the 45 participants the alignment could not be confirmed (R2<0.8) due to recording gaps or trigger alignment failures and they were disregarded from further analysis in social vs. non-social comparisons (kinematically adjusted), and in air touches vs. real touchscreen event categories. For further analysis, a threshold of 700 acceptable trials had to be met for the establishment of the SmRP and 350 trials (per category) in the cross-category comparisons. A key focus of our analysis was to compare social vs. non-social interactions. In addition to the instructions to diminish typing interactions we also determined that in the real world the typing interactions were dominated by intervals of 200 ms (median) and towards our analysis interactions separated by less than 3.5× threshold (700 ms) gap were excluded. The population median of median separations was 2 s (for both social and non-social interactions) during the laboratory testing. After these layers of exclusions, we were left with 34 participants for the establishment of SmRPs, 20 participants for the comparison between social vs. non-social interactions (without kinematic adjustments), and 24 participants for the comparison between air touches vs. real touchscreen events.

The EEG recordings were band-pass filtered between 0.1 to 70 Hz, and independent component analysis was run for subtracting blink artifacts from the EEG signals (Icablinkmetrics implemented in MATLAB). This was followed by another band-pass filter between 0.1 to 30 Hz (and a parallel set was created with 0.1 to 3 Hz focused on the slower signals). Trials above 1 mV were eliminated. The epoch durations were −4 s to 4 s from the touchscreen event, and signals were baseline corrected between −4 to −3 s from the touchscreen event. The signals were then processed using the hierarchical linear modelling toolbox LIMO EEG using ordinary least squares regression. The SmRP was tested using one sample t-test at the population level. For comparison of social and non-social touches, the trial-to-trial motor amplitudes were used towards an ANCOVA model at the single subject level and the resultant B values for the social and no-social categories were used towards the paired t-test at the population level. For comparison of air touches vs. real touchscreen events paired t-test was used at the population level. The statistics were for corrected for multiple comparison correction (MCC) using bootstrapped signals and 2-D spatiotemporal clustering implemented in LIMO EEG (α=0.05). The statistical masks of the main and parallel streams were merged using the logical ‘or’ operator. For the simple behavioural regression analysis robust (bi-square) linear regression was used.

### Subsequent Monitoring

Accordingly, it can be seen that patterns matching behavioural output with neuronal activity can be identified, along the lines above and illustrated as an example in FIG. 1. Such patterns can be stored in a database. For example, particular touches associated with particular smartphone functions can be matched to particular neuronal activities. These patterns can then be used, such as in the method of FIG. 2, to identify event-based neuronal activations based purely on the behavioural output. Accordingly, the smartphone can be used as a behavioural output recorder, such as with software (e.g. a background app) recording behavioural output of the user. Based upon such identified patterns as outlined hereabove, subsequent monitoring of users can be based solely on in loco smartphone use, recording behavioural outputs with a background app running on the smartphone. For example, a detection or recording of a smartphone touch by the background app allows a corresponding neuronal activity to be identified from the database of event-related neuronal activations. A change in identified event-related neuronal activations over a period of time may be associated with a particular function or area of the brain such that the change may be associated with a corresponding change in the function and/or area of the brain. The change may be associated with an impairment or disease and/or a treatment thereof-thereby enabling a monitoring of change in the user's brain.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims.

The applicant indicates that aspects of the present invention may consist of any such individual feature or combination of features. It should be understood that the embodiments described herein are merely exemplary and that various modifications may be made thereto without departing from the scope or spirit of the invention. For example, it will be appreciated that although shown here relating to a smartphone touch, in other examples other events, behaviours and associated neuronal activities are included.

What is claimed herein is:

1. A method of detecting a change in electrical signals in a brain of a user, the method comprising:
a) recording:
i) at least one electrical signal in the brain of a user by EEG; and
ii) at least one user behavior comprising a tap, a swipe, a gesture, or a sound input; wherein the recording is performed while the user is using a portable electronic device;
b) pattern matching the recorded at least one electrical signal and the recorded at least one user behavior with a database comprising:
i) individuals' electrical signals during portable electronic device use; and
ii) individuals' behaviors during portable electronic device use, wherein the individuals' behaviors comprise a tap, a swipe, a gesture, or a sound input; to identify the at least one electrical signal based on only the recorded at least one user behavior;
c) then recording, over a period of time with a portable electronic device, at least one user behavior comprising a tap, a swipe, a gesture, and a sound input, to detect a change in electrical signals in a brain of the user.

2. The method of claim 1, wherein the at least one electrical signal is an event-related potential.

3. The method of claim 2, wherein the event-related potential comprises a readiness potential (RP), a motor potential (MP), or a reafferent potential (RAP).

4. The method of claim 1, wherein the electrical signals are topographical electrical signals.

5. The method of claim 1, wherein the electrical signals are topographical event-related brain activity.

6. The method of claim 1, wherein the portable elective device is at least one of a smartphone and a smartwatch.

7. The method of claim 1, wherein the recording in step c) consists of unprompted smartphone use by the user outside of a laboratory or unprompted smartwatch use by the user outside of a laboratory.

8. A non-transitory computer readable carrier medium carrying computer readable code to carry out the method of claim 1.

9. A computer program product executable on a processor so as to implement the method of claim 1.

10. A non-transitory computer readable medium loaded with the computer program product of claim 9.

11. A processor arranged to implement the method of claim 1.

12. A system comprising a portable electronic device and the computer program product of claim 9 executable on a processor.
